(19) United States
(12) Patent Application Publication
(10) Pub. No.: US 2026/0240936 A1
(43) Pub. Date: Aug. 20, 2026

(54) GLUTAMINE FOR PRESERVING, IMPROVING OR RESTORING KIDNEY FUNCTION

(71) Applicant: Fresenius Kabi Deutschland GmbH, Bad Homburg (DE)

(72) Inventors: Martin Westphal, Bad Homburg (DE); John STOVER, Zurich (CH); Melanie BOTHE, Frankfurt (DE); Silke Baasner, Bad Homburg (DE); Rosa ABELE, Konigstein (DE)

(21) Appl. No.: 19/059,194

(22) Filed: Feb. 20, 2025

(51) Int. Cl. A61K 38/05; A61P 13/12

(52) U.S. Cl. CPC A61K 38/05; A61P 13/12

(57) ABSTRACT

The present disclosure relates to L-glutamine for use in preserving, improving or restoring kidney function or for decelerating and/or attenuating a decline in kidney function.

### FIELD OF THE INVENTION

The present invention relates to L-glutamine for improving kidney function.

### BACKGROUND OF THE INVENTION

L-glutamine is one of the 20 naturally occurring amino acids in dietary protein. The levels required are elevated during periods of disease and muscle wasting typical of physical trauma.

L-glutamine is sold as an isolated amino acid. It is also found in high levels in dietary meats and eggs. L-glutamine has been shown to improve immunity, to reduce inflammation, to regulate cellular hydration and to improve gut barrier function, specifically in metabolically stressed patients, e.g. in critically ill patients, surgical patients and bone marrow transplant patients.

Due to the fact that orally administered L-glutamine is metabolized to citrulline, L-alanine and other amino acids by the intestinal epithelial cells (Souba W W, Smith R J, Wilmore D W. Glutamine metabolism by the intestinal tract. JPEN J Parenter Enteral Nutr. 1985 September-October; 9(5):608-17.), only intravenous administration of L-glutamine allows for direct and predictable manipulation of plasma L-glutamine levels. Thus, advantageously, glutamine is provided in form of compositions for parenteral administration.

However, L-glutamine is poorly soluble in aqueous solvents.

Furthermore, due to the heat instability of L-glutamine, compositions comprising glutamine may not be terminally sterilized by autoclaving.

Therefore, and in order to increase chemical stability, solubility and shelf life, L-glutamine is mainly provided in the form of dipeptides. L-glutamine is released from the peptide by hydrolysis.

Commercially available compositions for parenteral use comprising L-glutamine in form of dipeptides are sold under the brand names Dipeptiven™ (concentrate providing L-glutamine in form of L-alanyl-L-glutamine) and Glamin™ (a balanced amino acid solution providing L-glutamine in form of glycyl-L-glutamine) respectively. The products are used in parenteral nutrition, specifically in patients with moderate to severe catabolic status.

It has long been known that patients with renal insufficiency should not receive nutrition too high in protein in order to prevent hyperuremia.

In a large clinical trial (Heyland D K, Elke G, Cook D, Berger M M, Wischmeyer P E, Albert M, Muscedere J, Jones G, Day A G; Canadian Critical Care Trials Group: Glutamine and Antioxidants in the critically ill patient: A post hoc analysis of a large-scale randomized trial. JPEN J Parenter Enteral Nutr. 2015 May; 39(4):401-9. doi: 0.1177/0148607114529994. Epub 2014 May 5.) it has recently been shown that acute renal failure is a contraindication for parenteral glutamine administration.

The use of Dipeptiven™ and Glamin™ respectively is contraindicated in patients with severe renal insufficiency, i.e. in patients with a creatinine clearance below 25 ml/minute. This is explicitly mentioned in the summary of product characteristics (SmPC) of both products. It would thus have been expected that L-glutamine may be harmful with respect to kidney function, particularly in patients with renal insufficiency.

### SUMMARY OF THE INVENTION

The present inventors have surprisingly found that providing L-glutamine parenterally not only does not impair but even supports kidney function.

### DETAILED DESCRIPTION OF THE INVENTION

The present disclosure relates to L-glutamine for use in preserving, improving or restoring kidney function or for decelerating and/or attenuating a decline in kidney function. L-glutamine is administered parenterally, preferably intravenously.

In the context of the present disclosure L-glutamine is provided in a composition which is administered parenterally. Preferably it is administered in the form of an aqueous solution. The composition may be a ready-to-use injectable liquid, a concentrate requiring dilution before administration or a powder for the preparation of a liquid composition for parenteral administration.

The composition providing L-glutamine may comprise L-glutamine as a free amino acid, as a dipeptide, as a tripeptide or as a tetrapeptide. In the context of the present disclosure, the composition preferably provides L-glutamine in form of a dipeptide, e.g. L-alanyl-L-glutamine or glycyl-L-glutamine. Most preferably, the composition provides L-glutamine in form of L-alanyl-L-glutamine.

When L-glutamine is provided in form of L-alanyl-L-glutamine, the composition suitably comprises 5 to 550 mg, preferably 10 to 400 mg, more preferably 20 to 250 mg L-alanyl-L-glutamine per ml.

When L-glutamine is provided in form of glycyl-L-glutamine, the composition suitably comprises 1 to 300 mg, preferably 5 to 200 mg, more preferably 10 to 100 mg glycyl-L-glutamine per ml.

In the context of the present disclosure the composition providing L-glutamine may provide L-glutamine or a source of L-glutamine, i.e. L-glutamine in form of di-, tri or tetrapeptide, as the only active ingredient.

However, it may also comprise further active ingredients.

It may for example comprise further amino acids, e.g. it may be a balanced amino acid solution.

The composition providing L-glutamine may further comprise pharmaceutically acceptable excipients, e.g. tonicity agents, agents for pH adjustment, antioxidants or antimicrobial agents.

### The Antioxidant

The composition may comprise at least one pharmaceutically acceptable antioxidant.

An antioxidant useful in the composition in the context of the disclosure may be any pharmaceutically acceptable compound having antioxidant activity, for example, the antioxidant may be selected form the group consisting of sodium metasulfite, sodium bisulfite, sodium sulfite, sodium thiosulfate, thioglycerol, thiosorbitol, thioglycolic acid, cysteine hydrochloride, n-acetly-cysteine, citric acid, alpha-tocopherol, beta-tocopherol, gamma-tocopherol, soluble forms of vitamin E, butylated hydroxyanisole (BHA), butylated hydroxytoluene (BHT), t-butylhydroquinone (TBHQ), monothioglycerol, propyl gallate, histidine, enzymes such as superoxide dismutase, catalase, selenium glutathione peroxidase, phospholipid hydroperoxide and glutathione peroxidase, Coenzyme Q10, tocotrienols, carotenoids, quinones, bioflavonoids, polyphenols, bilirubin, ascorbic acid, isoascorbic acid, uric acid, metal-binding proteins, ascorbic acid palmitate, an antioxidant obtained or obtainable from rosemary, rosemary extract and mixtures thereof.

If present, the total amount of agents with antioxidant activity is preferably in the range of from 0.001 wt. % to 0.05 wt. %, more preferably from 0.01 wt. % to 0.04 wt. %, more preferably from 0.01 wt. % to 0.03 wt. %, and even more preferably from 0.015 wt. % to 0.025 wt. % based on the total weight of the composition.

### The Tonicity Agent

The composition in the context of the present disclosure may comprise at least one pharmaceutically acceptable tonicity agent.

Tonicity agents are used to confer tonicity. Suitable tonicity agents may be selected from the group consisting of sodium chloride, mannitol, lactose, dextrose, sorbitol and glycerol.

Preferably, the total amount of tonicity agents is in the range of 0.1 to 10 wt. %, more preferably from 1 wt. % to 5 wt. %, more preferably from 1 wt. % to 4 wt. %, more preferably 1 wt. % to 3 wt. %, more preferably from 1.5 wt. % to 2.8 wt. %, and even more preferably from 2.0 wt. % to 2.8 wt. % based on the total weight of the emulsion.

Preferably, the composition as parenterally administered has an osmolality in the range of from 200 to 900, more preferably of from 250 to 600, most preferably of from 300 to 450 mOsmol/kg, measured with a Vapor Pressure Osmometer, Model 5520 (Vapro™) according to USP <785>.

### pH Adjustment

The pH of the composition may be adjusted by adding solutions of conventionally known acids or bases such as HCl and NaOH or through the use of buffers, such as phosphate or citrate buffers.

The final pH of the composition is preferably in the range of from 5.5 to 7.5.

### The Antimicrobial Agent

The composition may comprise at least one pharmaceutically acceptable antimicrobial agent.

An antimicrobial agent useful in the composition in the context of the present disclosure may be any pharmaceutically acceptable compound having antimicrobial activity, for example, the antimicrobial agent may be selected form the group consisting of benzalkonium chloride, benzethonium chloride, benzyl alcohol, chlorobutanol, chlorocresol, cresol, parabens (methyl, ethyl, propyl, butyl esters), phenol, phenylmercuric nitrate and thimerosol.

If present, the total amount of agents with antimicrobial activity is preferably in the range of from 0.001 wt. % to 2.5 wt. %, more preferably from 0.005 wt. % to 1.5 wt. %, more preferably from 0.01 wt. % to 1.1 wt. %, and even more preferably from 0.01 wt. % to 0.5 wt. % based on the total weight of the composition.

It is to be understood that the composition providing L-glutamine, in order to be suitable for parenteral administration, has to be sterile and pyrogen-free which is accomplished by aseptic manufacturing and filling techniques and/or by terminally sterilizing the compositions in an autoclave.

The composition may be manufactured according to standard methods known in the art, e.g. by dissolving the ingredients in a suitable solvent or solvent mixture. The resulting solution may optionally be lyophilized.

The composition providing L-glutamine is administered parenterally as a medicament.

However, the composition providing L-glutamine may also be mixed with other compositions, e.g. with lipid emulsions, amino acid solutions and carbohydrate solutions in the context of a parenteral nutrition regimen.

It may also be administered as a parenteral supplement to an enteral nutrition regimen.

The present disclosure includes inter alia the following aspects: In a first aspect the present disclosure relates to L-glutamine for use in preserving, improving or restoring kidney function or for decelerating and/or attenuating a decline in kidney function, wherein L-glutamine is provided in a composition that is administered parenterally, preferably intravenously.

In a second aspect the present disclosure relates to L-glutamine for use according to aspect 1, wherein improving or restoring kidney function or decelerating and/or attenuating a decline in kidney function involves the treatment or prevention of chronic renal disease, preferably chronic progressive nephropathy.

In a third aspect the present disclosure relates to L-glutamine for use according to aspect 1 or 2, wherein improving or restoring kidney function or decelerating and/or attenuating a decline in kidney function involves the treatment or prevention of glomerulosclerosis and/or tubular basophilia and/or tubular atrophy and/or thickening of the tubular basement membrane.

In a fourth aspect the present disclosure relates to L-glutamine for use according to any of aspects 1 to 3, wherein the treatment involves the deceleration and/or attenuation of the development and/or the reversal of glomerulosclerosis and/or tubular basophilia and/or tubular atrophy and/or thickening of the tubular basement membrane.

In a fifth aspect the present disclosure relates to L-glutamine for use according to any of aspects 1 to 4 in patients with renal insufficiency.

In a sixth aspect the present disclosure relates to L-glutamine for use according to aspect 5, wherein the renal insufficiency involves a reduced glomerular filtration rate (GFR).

In a seventh aspect the present disclosure relates to L-glutamine for use according to aspect 5 or 6, wherein the renal insufficiency involves a creatinine clearance of 15 to 89 ml per minute.

In an eighth aspect the present disclosure relates to L-glutamine for use according to any of aspects 5 to 7, wherein the renal insufficiency involves a creatinine clearance of 60 to 89 ml per minute.

In a ninth aspect the present disclosure relates to L-glutamine for use according to any of aspects 5 to 7, wherein the renal insufficiency involves a creatinine clearance of 45 to 59 ml per minute.

In a tenth aspect the present disclosure relates to L-glutamine for use according to any of aspects 5 to 7, wherein the renal insufficiency involves a creatinine clearance of 25 to 44 ml per minute.

In an eleventh aspect the present disclosure relates to L-glutamine for use according to any of the preceding aspects in patients suffering from any of diabetes, ischemia, infection, intoxication, hypertension, sepsis, focal segmental glomerulosclerosis, reflux nephropathy, sickle cell disease, autoimmune disease, malnutrition, cachexia, inflammation, hyperuricemia, and/or in patients admitted to an intensive care unit and/or in trauma patients and/or in post-surgical patients.

In a twelfth aspect the present disclosure relates to L-glutamine for use according to any of the preceding aspects, wherein the daily glutamine dose is 50 to 5000, preferably 100 to 4500 mg per kg bodyweight.

In a thirteenth aspect the present disclosure relates to L-glutamine for use according to any of the preceding aspects, wherein the composition provides 5 to 500 mg, preferably 10 to 300 mg, more preferably 15 to 150 mg L-glutamine per ml.

In a fourteenth aspect the present disclosure relates to L-glutamine for use according to any of the preceding aspects, wherein the composition comprises L-glutamine as a free amino acid or in form of a dipeptide, tripeptide or tetrapeptide, preferably in form of a dipeptide, more preferably in form of L-alanyl-L-glutamine or glycyl-L-glutamine.

In a fifteenth aspect the present disclosure relates to L-glutamine for use according to any of the preceding aspects, wherein the composition comprises 5 to 550 mg, preferably 10 to 400 mg, more preferably 20 to 250 mg L-alanyl-L-glutamine per ml.

In a sixteenth aspect the present disclosure relates to L-glutamine for use according to any of the aspects 1 to 14, wherein the composition comprises 1 to 300 mg, preferably 5 to 200 mg, more preferably 10 to 100 mg glycyl-L-glutamine per ml.

### EMBODIMENTS

1) L-glutamine for use in preserving, improving or restoring kidney function or for decelerating and/or attenuating a decline in kidney function, wherein L-glutamine is provided in a composition that is administered parenterally, preferably intravenously.
2) L-glutamine for use according to embodiment 1, wherein the preserving, improving or restoring kidney function or the decelerating and/or attenuating a decline in kidney function involves the treatment or prevention of chronic renal disease, preferably chronic progressive nephropathy.
3) L-glutamine for use according to embodiment 1 or 2, wherein the preserving, improving or restoring kidney function or the decelerating and/or attenuating a decline in kidney function involves the treatment or prevention of glomerulosclerosis and/or tubular basophilia and/or tubular atrophy and/or thickening of the tubular basement membrane.
4) L-glutamine for use according to embodiment 2 or 3, wherein the treatment involves the deceleration and/or attenuation of the development and/or the reversal of glomerulosclerosis and/or tubular basophilia and/or tubular atrophy and/or thickening of the tubular basement membrane.
5) L-glutamine for use according to any of the preceding embodiments in patients with renal insufficiency.
6) L-glutamine for use according to claim 5, wherein the renal insufficiency involves a reduced glomerular filtration rate (GFR).
7) L-glutamine for use according to embodiment 5 or 6, wherein the renal insufficiency involves a creatinine clearance of 15 to 89 ml per minute.
8) L-glutamine for use according to any of embodiments 5 to 7, wherein the renal insufficiency involves a creatinine clearance of 60 to 89 ml per minute.
9) L-glutamine for use according to any of claims 5 to 7, wherein the renal insufficiency involves a creatinine clearance of 45 to 59 ml per minute.
10) L-glutamine for use according to any of embodiments 5 to 7, wherein the renal insufficiency involves a creatinine clearance of 25 to 44 ml per minute.
11) L-glutamine for use according to any of the preceding embodiments in patients suffering from any of diabetes, ischemia, infection, intoxication, hypertension, sepsis, focal segmental glomerulosclerosis, reflux nephropathy, sickle cell disease, autoimmune disease, malnutrition, cachexia, inflammation, hyperuricemia, and/or in patients admitted to an intensive care unit and/or in trauma patients and/or in post-surgical patients.
12) L-glutamine for use according to any of the preceding embodiments, wherein the daily glutamine dose is 50 to 5000, preferably 100 to 4500 mg per kg bodyweight.
13) L-glutamine for use according to any of the preceding embodiments, wherein the composition provides 5 to 500 mg, preferably 10 to 300 mg, more preferably 15 to 150 mg L-glutamine per ml.
14) L-glutamine for use according to any of the preceding embodiments, wherein the composition comprises L-glutamine as a free amino acid or in form of a dipeptide, tripeptide or tetrapeptide.
15) L-glutamine for use according to any of the preceding embodiments, wherein the composition comprises L-glutamine in form of a dipeptide.
16) L-glutamine for use according to embodiment 15, wherein the dipeptide is L-alanyl-L-glutamine or glycyl-L-glutamine.
17) L-glutamine for use according to any of the preceding embodiments, wherein the composition comprises 5 to 550 mg, preferably 10 to 400 mg, more preferably 20 to 250 mg L-alanyl-L-glutamine per ml.
18) L-glutamine for use according to any of the preceding embodiments, wherein the composition comprises 1 to 300 mg, preferably 5 to 200 mg, more preferably 10 to 100 mg glycyl-L-glutamine per ml.
19) L-glutamine for use according to any of the embodiments 1 to 17, wherein the composition is an aqueous solution of L-alanyl-L-glutamine.
20) L-glutamine for use according to embodiment 17 or 19, wherein the composition is a concentrate that has to be diluted before being parenterally administered.
21) L-glutamine for use according to embodiment 20, wherein the concentrate consists of water and L-alanyl-L-glutamine.
22) L-glutamine for use according to any of embodiments 17 or 19 to 21, wherein the composition comprises 150 to 250 mg L-glutamine per ml.
23) L-glutamine for use according to any of the embodiments 1 to 16 or 18, wherein the composition is a ready-to-use balanced amino acid solution.
24) Method for preserving, improving or restoring kidney function or for decelerating and/or attenuating a decline in kidney function comprising the parenteral administration of an effective dose of L-glutamine, wherein L-glutamine is provided by a composition according to any of embodiments 13 to 23.
25) Method according to embodiment 24, wherein the preserving, improving or restoring kidney function or the decelerating and/or attenuating a decline in kidney function involves the treatment or prevention of chronic renal disease, preferably chronic progressive nephropathy.
26) Method according to embodiment 24 or 25, wherein the preserving, improving or restoring kidney function or the decelerating and/or attenuating a decline in kidney function involves the treatment or prevention of glomerulosclerosis and/or tubular basophilia and/or tubular atrophy and/or thickening of the tubular basement membrane.
27) Method according to embodiment 25 or 26, wherein the treatment involves the deceleration and/or attenuation of the development and/or the reversal of glomerulosclerosis and/or tubular basophilia and/or tubular atrophy and/or thickening of the tubular basement membrane.
28) Method according to any of embodiments 24 to 27 in patients according to any of embodiments 5 to 11.
29) Method according to any of embodiments 24 to 28, wherein the daily dose of L-glutamine is 50 to 5000, preferably 100 to 4500 mg.

### EXAMPLES

The effect of L-glutamine (administered in form of L-alanyl-L-glutamine; Dipeptiven™) following daily continuous intravenous infusion was examined in a 5/6 kidney nephrectomised rat model.

L-alanyl-L-glutamine was administered for 9 consecutive days. The effects attributable to L-glutamine (provided in form of alanyl-glutamine; Dipeptiven™) were determined by comparison to an L-alanine infusion.

To prevent protein overdose, the protein content of the animal food was adapted according to the L-alanyl-L-glutamine or L-alanine dose administered intravenously.

### Experimental Procedures

The study was conducted according to the following design:

| | | | | | Dose | Diet | |
|---|---|---|---|---|---|---|---|
| | | Dose level | Dose volume | | concentration | Protein | Number |
| Group | Treatment | (mg/kg/day) | (mL/kg/day) | (mL/kg/h) | (mg/mL) | % | of males |
| 2. Control 2 | Vehicle | 0 | 37.5 | 1.5625 | 0 | 12 | 10 |
| 3. 0.5 Ala-Gln | Dipeptiven ™ | 500 | 37.5 | 1.5625 | 13.33 | 12 | 10 |
| 4. 3.0 Ala-Gln | Dipeptiven ™ | 3000 | 37.5 | 1.5625 | 80 | 9 | 10 |
| 5. 7.5 Ala-Gln | Dipeptiven ™ | 7500 | 37.5 | 1.5625 | 200 | 4.5 | 10 |
| 6. 7.5 Ala-Gln | Dipeptiven ™ | 7500 | 37.5 | 1.5625 | 200 | 12 | 10 |
| 7. 1.2 Ala | Alanine | 1200 | 37.5 | 1.5625 | 32 | 10.8 | 10 |
| 8. 3.0 Ala | Alanine | 3000 | 37.5 | 1.5625 | 80 | 9 | 10 |

Ala-Gln = L-alanyl-L-glutamine; N(2)-L-alanyl-L-glutamine
Ala = L-alanine

Group 1 and 2 animals (control) received the vehicle (0.9% NaCl).

Groups 5 and 6 received Dipeptiven™ without any dilution, i.e. L-alanyl-L-glutamine in a concentration of 200 mg/ml.

Group 3 and 4 animals received diluted Dipeptiven™, i.e. L-alanyl-L-glutamine in a concentration of 13.33 and 80 mg/ml respectively.

Group 7 and 8 animals received L-alanine in a concentration of 32 and 80 mg/ml respectively.

All animals in groups 1 and 3 to 8 underwent a 5/6 nephrectomy. All animals in group 2 were “sham operated”, meaning the abdomen was opened and closed in the surgery, but the kidneys were not excised and remained in the body.

During the acclimatisation period, a polyurethane catheter was implanted into the posterior vena cava via the left femoral vein. Following implantation, the animals were maintained on continuous infusion with physiological saline prior to the start of treatment.

Morbidity/mortality checks were performed at least twice daily. Clinical observations were performed daily. A full clinical examination was performed daily. Individual body weights were recorded three times a week. Food consumption was measured three times a week for each cage of animals, including one measurement during the acclimatisation period. Clinical laboratory determinations were performed on selected animals on days −1 and on days 1 to 10. Animals were sampled for amino acid determination on days 1, 4, 7 and 10. Animals were fasted before blood sampling performed on day 10.

All animals were killed at the end of the treatment period (day 10) and necropsied. Organ/tissue samples were fixed and preserved at necropsy for all animals. Selected organs/tissues from all animals were examined histopathologically. In addition, selected organs/tissues from all animals were used for amino acid determination.

### Results

No treatment-related clinical signs or changes in blood levels of biomarkers for kidney and liver injury were noted throughout the treatment period for animals treated with L-alanyl-L-glutamine and for those treated with L-alanine whatever the dose and diet. The intravenous infusion of L-alanyl-L-glutamine and L-alanine respectively for 9 days in a 5/6 kidney nephrectomised rat model was overall well tolerated.

The infusion of L-alanyl-L-glutamine led to a decreased incidence of chronic progressive nephropathy.

In detail, the intravenous administration of L-alanyl-L-glutamine to nephrectomised rats by continuous infusion over a period of 9 days in doses of up to 7500 mg/kg/day or L-alanine in doses of up to 3000 mg/kg/day led to the following test items related changes in the incidence of chronic progressive nephropathy: Control: 3/5 animals, L-alanyl-L-glutamine 0.5 g/kg/day: 2/5 animals, L-alanyl-L-glutamine 3.0 g/kg/day: 0/5 animals, L-alanyl-L-glutamine 7.5 g/kg/day: 0/5 animals, L-alanyl-L-glutamine 7.5 g/kg/day (+12% protein in diet): 2/5 animals, L-alanine 1.2 g/kg/day: 4/5 animals, L-alanine 3.0 g/kg/day: 3/5 animals. These results are depicted in the table below:

| | | | Group 3 | Group 4 | Group 5 | Group 6 | Group 7 | Group 8 |
|---|---|---|---|---|---|---|---|---|
| | Group 1 | Group 2 | 0.5 g/kg | 3.0 g/kg | 7.5 g/kg | 7.5 g/kg | 1.2 g/kg | 3.0 g/kg |
| | control | sham | Ala-Gln | Ala-Gln | Ala-Gln | Ala-Gln | Ala | Ala |
| Protein in diet | 12% | 12% | 12% | 9% | 4.5% | 12% | 10.8% | 9% |
| Nephropathy | 3/5 | 0/5 | 2/5 | 0/5 | 0/5 | 1/5 | 4/5 | 3/5 |

Except for group 2 (“sham” operated) animals, all animals underwent 5/6 nephrectomy, meaning 5/6 of the kidney tissue was removed. During the treatment period, all animals received the recommended amount of protein for this age group, namely 12% of protein in the diet.

In the groups receiving L-alanyl-L-glutamine or L-alanine, extra amino acids were supplied intravenously in form of a L-alanyl-L-glutamine (Dipeptiven™) or L-alanine respectively. To prevent an excess supply of amino acids i.e. an excess supply of nitrogen, the amount of protein in the diet was reduced accordingly.

In group 1 the animals underwent nephrectomy and received saline plus 12% protein in the diet. 60% (3/5) of these animals developed a chronic progressive nephropathy.

In group 2, the animals retained their kidneys and received saline plus 12% protein the diet. None of these animals developed a chronic progressive nephropathy.

All animals in groups 3 to 8 underwent nephrectomy and were treated with either L-alanyl-L-glutamine or L-alanine. L-alanyl-L-glutamine treatment (in groups 3, 4, and 5) reduced the occurrence of chronic progressive nephropathy dose-dependently from 60% to 40% to 0%, while L-alanine treatment did not.

Of note, animals in group 6 received both a high dose of glutamine and 12% protein in the diet, thus an amino acid overload. Amino acid/protein/nitrogen overload is known to increase the occurrence of chronic progressive nephropathy. Still only 20% (1/5) of the animals in this group developed chronic progressive nephropathy.

The increasing doses of glutamine were adjusted for by decreasing the dosages of protein in the diet in groups 3, 4, and 5 respectively.

Low protein diet is known to reduce the occurrence of chronic progressive nephropathy and thus might be suggested as the cause of the beneficial effect.

This is, however, easily disproven by comparing groups 4 and 8. Both groups received a 9% protein diet. Group 4 animals were treated with L-alanyl-L-glutamine and no chronic nephropathy occurred, while group 8 animals were treated with L-alanine and 60% of the animals developed a chronic progressive nephropathy. Thus, the beneficial effect is not due to the decreased amount of protein in the diet, but due to the L-alanyl-L-glutamine treatment.

These experiments clearly demonstrate that parenteral administration of L-glutamine reduces the occurrence of chronic progressive nephropathy in a dose-dependent manner.

1-15. (canceled)

16. A method for treating renal disease in a patient, said method comprising administering a parenteral composition comprising L-glutamine as a dipeptide, as a tripeptide or as a tetrapeptide;
wherein said composition preserves, improves or restores kidney function, or decelerates and/or attenuates a decline in kidney function in the patient;
wherein the treatment of chronic renal disease involves the treatment of glomerulosclerosis and/or tubular basophilia and/or tubular atrophy and/or thickening of the tubular basement membrane, and provides 50 to 500 mg L-glutamine per mL; and
wherein the daily glutamine dose is 50 to 5000 mg per kg bodyweight.

17. The method according to claim 16, wherein said patient has renal insufficiency.

18. The method according to claim 17, wherein the renal insufficiency involves a reduced glomerular filtration rate (GFR).

19. The method according to claim 18, wherein the renal insufficiency involves a creatinine clearance of 15 to 89 ml per minute.

20. The method according to claim 19, wherein the renal insufficiency involves a creatinine clearance of 60 to 89 ml per minute.

21. The method according to claim 16, wherein said patient suffers from any of diabetes, ischemia, infection, intoxication, hypertension, sepsis, focal segmental glomerulosclerosis, reflux nephropathy, sickle cell disease, autoimmune disease, malnutrition, cachexia, inflammation, hyperuricemia, and/or in patients admitted to an intensive care unit and/or in trauma patients and/or in post-surgical patients.

22. The method according to claim 16, wherein said composition is provided intravenously.

23. The method according to claim 16, wherein said composition further comprises one or more pharmaceutically acceptable excipients.

24. The method according to claim 16 wherein said pharmaceutically acceptable excipient is chosen from tonicity agents, agents for pH adjustment, antioxidants and antimicrobial agents.

25. The method according to claim 16, wherein said composition is in the form of an aqueous solution.

26. The method according to claim 16, wherein said composition is a ready-to-use injectable liquid, or a concentrate requiring dilution before administration.

27. The method according to claim 18, wherein the renal insufficiency involves a creatinine clearance of 15 to 89 ml per minute,
wherein the composition comprises L-glutamine is in the form of L-alanyl-L-glutamine or glycyl-L-glutamine.

28. The method according to claim 16, wherein the composition comprises L-glutamine is in the form of L-alanyl-L-glutamine or glycyl-L-glutamine.

29. The method according to claim 16, wherein the composition comprises 5 to 550 mg L-alanyl-L-glutamine per ml.

30. A method for treating chronic renal disease in a patient, said method comprising measuring the kidney function of the patient; and
administering a parenteral composition comprising L-glutamine as a dipeptide, as a tripeptide or as a tetrapeptide;
wherein the treatment of chronic renal disease involves preserving, improving or restoring kidney function or decelerating and/or attenuating a decline in kidney function.

31. The method of claim 30, wherein measuring the kidney function of the patient comprises measuring the kidney function of the patient after the administering the parenteral composition comprising L-glutamine as a dipeptide, as a tripeptide or as a tetrapeptide.

32. The method of claim 30, wherein measuring the kidney function of the patient comprises measuring the kidney function of the patient at a first time before the administering the parenteral composition and at a second time after the administering the parenteral composition.
